(19) United States
(12) Patent Application Publication
(10) Pub. No.: US 2015/0320696 A1
(43) Pub. Date: Nov. 12, 2015

(54) COMBINATION THERAPY FOR CANCER

(71) Applicants: Peter TIROSH, Petah Tikva (IL); Shlomo PLEBAN, Kiriat Ono (IL)

(72) Inventors: Peter TIROSH, Netanya (IL); Shlomo PLEBAN, Kiriat Ono (IL)

(21) Appl. No.: 14/650,867

(22) PCT Filed: Dec. 10, 2013
(86) PCT No.: PCT/IL13/51021

§ 371 Date: Jun. 9, 2015

Related U.S. Application Data
(60) Provisional application No. 61/735,575, filed on Dec. 11, 2012.

(51) Int. Cl. A61K 31/045; A61K 39/395; C07K 16/30; A61K 31/282; A61K 31/7068

(52) U.S. Cl. CPC A61K 31/045; A61K 31/282; A61K 31/7068; C07K 16/30; A61K 39/39558

(57) ABSTRACT

The present invention relates to a combination therapy for the treatment of cancer, particularly to combinations of terpinen-4-ol and at least one additional anti-cancer therapeutic agent. The combination therapy of the present invention shows enhanced anti-cancerous therapeutic effects compared to the effect of each of the components administered alone. In some embodiments, the combination therapy provide for a synergistic anti-cancer effect.

### FIELD OF THE INVENTION

The present invention relates to a combination therapy for the treatment of cancer, particularly to combinations of terpinen-4-ol and at least one additional anti-cancer therapeutic agent, wherein the combination therapy shows enhanced anti-cancer effect.

### BACKGROUND OF THE INVENTION

Monoterpenes are major secondary metabolites present in plants, known to be associated with the plant defense mechanisms against biotic stress. The monoterpenes consist of two isoprene units and are found in significant amounts in essential oils. In addition, numerous monoterpenes have been proposed to exert potent antitumor action, and some have shown promising results in the prevention and treatment of a variety of cancers in tumor model systems. Notably, two naturally occurring monoterpenes, perillyl alcohol (POH) and limonene (LIM), are currently undergoing clinical trials to evaluate their therapeutic effect.

Terpinen-4-ol is a naturally occurring monoterpene found in the essential oils of many aromatic plants including Melaleuca alternifolia (tea tree oil), various types of Eucalyptus and Alpinia zerumbet (shell ginger). Terpinen-4-ol is used as an anti-inflammatory and antioxidant agent (for example, Carson C F et al., 2006. Clinical Microbiology Reviews 19(1):50-62) and has been shown to have antiviral, antibacterial, antifungal, and insecticidal effects.

Terpinen-4-ol has also been shown to impair the growth of human M14 melanoma cells. It appears to be more effective on the drug (adriamicin)-resistant cell line (ADR), which express high levels of P-glycoprotein in the plasma membrane, overcoming resistance to caspase-dependent apoptosis exerted by P-glycoprotein-positive tumor cells (Calcabrini A et al., 2004. J Invest Dermatol 122:349-360). Recent reports have been shown that this compound also exhibits significant anti-proliferative effects against murine AE17 mesothelioma and B16 melanoma tumor cell lines. It has been demonstrated that terpinen-4-ol caused primary necrotic cell death and cell cycle arrest of these aggressive tumor cells (Greay S J et al., 2010. Cancer Chemother Pharmacol 65:877:888).

U.S. Patent Application Publication No. 2002/0141952 discloses various compounds including alcohols, diols and/or triols and their analogues, terpinen-4-ol being one compound among many others, used in compositions and methods for regulating the melanin content of mammalian melanocytes; regulating pigmentation in mammalian skin, hair, wool or fur; treating or preventing various skin and proliferative disorders.

U.S. Patent Application Publication No. 2008/0015249 discloses pharmaceutical compositions containing plant essential oils, natural or synthetic, or mixtures or derivatives thereof terpinen-4-ol being one of them, for the prevention and treatment of soft tissue cancer in mammals.

International Patent Application Publication No. WO 2010/006376 discloses compositions for the treatment and/or prevention of cancer, comprising a therapeutically effective amount of tea tree oil from Melaleuca alternifolia, or its derivatives, including terpinen-4-ol. Methods for the treatment and/or prevention of cancer are also described, particularly for the treatment and prevention of basal cell carcinoma, squamous cell carcinoma and/or melanoma.

International Patent Application Publication No. WO 2012/104569 discloses composition of monoterpenoids for the treatment of cancer. Among the monoterpenoid disclosed are α-terpineol, β-terpineol, γ-terpineol, terpinen-4-ol, menthol, thymol, carvacrol, carveol, perillyl alcohol, isopulegol, limonene-10-ol, and dihydrocarveol.

Cancer or neoplasm is a malignant growth characterized by unregulated proliferation of cells. Cancerous cells propagate from a single cell and can be multiplied to develop into tumor tissues. The cancerous cells can invade nearby tissues and spread through the bloodstream and lymphatic system to other parts of the body (metastasis). Most cancers can be treated, and some cured, depending on the specific type, location, and stage of development. Once diagnosed, cancer is usually treated with one or a combination of surgery, chemotherapy and radiotherapy.

Surgery generally is only effective for treating the earlier stages of cancer and in removing tumors located at certain sites, for example, in the breast, colon, and skin. However, it cannot be used in the treatment of tumors located in other areas inaccessible to surgeons, nor in the treatment of disseminated neoplastic conditions such as leukemia.

Radiation therapy is only effective for treating clinically localized disease at early and middle stages of cancer, and is not effective for the late stages of cancer with metastasis. Radiation is generally applied to a defined area of the subject's body which contains abnormal proliferative tissue, in order to maximize the dose absorbed by the abnormal tissue and minimize the dose absorbed by the nearby normal tissue. However, it is difficult (if not impossible) to selectively administer therapeutic radiation to the abnormal tissue. Thus, normal tissue proximate to the abnormal tissue is also exposed to potentially damaging doses of radiation throughout the course of treatment.

The majority of chemotherapeutic drugs can be divided into: alkylating agents (e.g. cyclophosphamide), antimetabolites (e.g. fluorouracil), plant alkaloids (e.g. paclitaxel), topoisomerase inhibitors (e.g. topotecan), and cytotoxic antibiotics (e.g. daunorubicin). All of these drugs impair cell division or DNA synthesis and functions. However, most of the chemotherapeutic drugs cause undesirable systemic effects such as cardiac or renal toxicity, marrow aplasia, alopecia, nausea and vomiting.

Immunotherapy is now an emerging treatment modality for a variety of cancers and several promising treatments have been already approved and are being tested in clinical trials. Antibodies are useful in cancer therapy because they can recognize tumor-associated antigens expressed at higher density on malignant compared to normal cells Immunotherapy can be used as a single therapy or in combination with traditional drug therapies. In the past two decades antibodies have been the fastest growing class of pharmaceutical proteins.

The shortcomings of the presently available cancer therapies have lead to the search for combinations treatments that may answer at least some of these shortcomings. For example, U.S. Patent Application Publication No. 2003/0108623 discloses pharmaceutical compositions containing plant essential oil compounds including terpinen-4-ol or mixtures or derivatives thereof, with one or more signal transduction modulators, for the prevention and treatment of cancer. The pharmaceutical composition can be administered with a conventional cancer treatment, e.g., tamoxifen.

U.S. Patent Application Publication No. 2004/0092583 discloses the use of incensole and/or furanogermacrens, derivatives, metabolites and precursors thereof in the treatment of neoplasia, particularly resistant neoplasia and immunodysregulatory disorders. The compounds, terpinen-4-ol being one among many others, can be administered alone or in combination with conventional chemotherapeutic, anti-rival or anti-parasite agents, and further in combination with radiation and/or surgery.

U.S. Patent Application Publication No. 2008/0113042 discloses pharmaceutical compositions and methods for cancer treatment based on combinational use of conventional anticancer agents and geranium oil or compounds thereof. The compositions are disclosed to be effective in broad range of cancer types.

Due to the severity and breadth of cancer diseases, there is a recognized need for additional effective means and methods for treating cancer with improved outcome.

### SUMMARY OF THE INVENTION

The present invention provides a combination therapy for treating various types of cancer. Particularly, the present invention provides compositions and methods combining terpinen-4-ol and at least one additional cancer therapy that show significant enhancement of the anti-cancer effect, which is preferably synergistic.

The present invention is based in part on the unexpected discovery that terpinen-4-ol and various anti-cancer agents show a synergistic inhibiting effect on the proliferation of various cancerous cell types. This phenomenon was observed in a wide range of cancer cell lines representing different types of cancer, and further in an in vivo model of colorectal cancer.

Without wishing to be bound by any particular theory or mechanism of action this synergistic effect may be attributed to the capability of terpinen-4-ol to significantly enhance the effect of known anti-cancer agents, including chemotherapeutic drugs as well as biologic drugs, particularly antibodies. Such combinations may therefore be used for treating wide range of cancers.

The combination therapy is particularly advantageous, since not only the anti-cancerous effect is enhanced compared to the effect of each compound alone, the dosage of each agent in a combination therapy can be reduced as compared to monotherapy with each agent, while still achieving an overall anti-tumor effect. In addition, due to the synergistic effect, the total amount of drugs administered to a patient can advantageously be reduced, which may result in decreased side effects.

Thus, according to one aspect, the present invention provides a method for treating cancer, the method comprising administering to a subject in need thereof (a) an effective amount of terpinen-4-ol and (b) an effective amount of at least one additional anti-cancer agent to provide a combination therapy having an enhanced therapeutic effect compared to the effect of the terpinen-4-ol and the at least one additional anti-cancer agent each administered alone. According to certain exemplary embodiments, the combination therapy has a synergistic therapeutic effect. According to this embodiment, the combination therapy produces a significantly better anti-cancer result (e.g., cell growth arrest, apoptosis, induction of differentiation, cell death, etc.) than the additive effects achieved by each individual constituent when administered alone at a therapeutic dose.

According to certain embodiments, the cancer is a solid tumor. According to other embodiments, the cancer is a non-solid tumor.

According to some embodiments, the solid-tumor cancer is selected from the group consisting of tumors of the central nervous system, breast cancer, prostate cancer, skin cancer (including basal cell carcinoma, cell carcinoma, squamous cell carcinoma and melanoma), cervical cancer, uterine cancer, lung cancer, ovarian cancer, testicular cancer, thyroid cancer, astrocytoma, glioma, pancreatic cancer, mesotheliomas, gastric cancer, liver cancer, colon cancer, rectal cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas and rhabdomyosarcomas. Each possibility represents a separate embodiment of the present invention.

According to other embodiments, the non-solid tumor is a blood cancer, including, for example, leukemia and lymphoma. Each possibility represents a separate embodiment of the present invention.

According to certain exemplary embodiments, the cancer is selected from the group consisting of colorectal cancer, gastric cancer, pancreatic cancer and prostate cancer. Each possibility represents a separate embodiment of the present invention. According to certain embodiments, the at least one additional anti-cancer agent is a chemotherapeutic agent. Suitable chemotherapeutic agents include, but are not limited to, alkylating agents, antibiotic agents, antimetabolic agents, hormonal agents, plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors, gene therapy agents, cell therapy, e.g., stem cells, or any combination thereof.

According to these embodiments, the chemotherapeutic agent is selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof. Each possibility represents a separate embodiment of the invention.

According to certain embodiments, the at least one additional agent is a biologic drug, particularly an antibody. According to some embodiments, the antibody is selected from the group consisting of cetuximab, anti-CD24 antibody, panitumumab and bevacizumab.

According to certain embodiments, the additional anti-agent cancer is known to be effective in treating a particular type of cancer.

According to some embodiments, the cancer is gastrointestinal cancer and the at least one additional anti-cancer agent is selected from the group consisting of oxaliplatin (Eloxatin®), fluorouracil (5-FU), anti-CD24 antibody, cetuximab (Erbitux®), irinotecan, panitumumab (Vectibix®), cisplatin, S-1 (dihydropyrimidine dehydrogenase (DPD) inhibitory fluoropyrimidine) and bevacizumab (Avastin®). Each possibility represents a separate embodiment of the present invention.

According to other embodiments, the cancer is pancreatic cancer, and the at least one additional anti-cancer agent is selected from the group consisting of gemcitabine (Gemzar®), erlotinib hydrochloride (Tarceva®) or GemCap (a combination of gemcitabine and capecitabine), and humanized anti-CD24 monoclonal antibodies. Each possibility represents a separate embodiment of the present invention.

According to yet additional embodiments, the cancer is prostate cancer and the at least one additional anti-cancer agent is selected from the group consisting of cetuximab (Erbitux®), bevacizumab (Avastin®) and humanized anti-CD24 monoclonal antibody. Each possibility represents a separate embodiment of the present invention.

The terms “combination therapy” or “combined treatment” or “in combination” as used herein denotes any form of concurrent or parallel treatment with at least two distinct therapeutic agents.

According to certain embodiments, the terpinen-4-ol and the at least one additional anti-cancer agent are administered simultaneously, either in the same composition or in separate compositions. According to other embodiments, the terpinen-4-ol and the at least one additional anti-cancer agent are administered sequentially, i.e., the terpinen-4-ol is administered either prior to or after the administration of the additional anti-cancer agent. In some embodiments, the administration of the terpinen-4-ol and the additional anti-cancer agent are concurrent, i.e., the administration period of the terpinen-4-ol and that of the agent overlap with each other. In some embodiments, the administration of the terpinen-4-ol and the additional anti-cancer agent are non-concurrent. For example, in some embodiments, the administration of the terpinen-4-ol is terminated before the additional agent is administered. In some embodiments, the administration of the additional anti-cancer agent is terminated before the terpinen-4-ol is administered.

According to certain typical embodiments, the terpinen-4-ol and the additional anti-cancer agent are administered within a single therapeutic composition. According to some embodiments, the therapeutic composition further comprises therapeutically acceptable diluents or carrier.

According to certain embodiments, the terpinen-4-ol is administered in an amount of from 0.1 mg/Kg body weight to 100 mg/Kg body weight. According to other embodiments, the terpinen-4-ol is administered at an amount of from 0.5 mg/Kg body weight to 20 mg/Kg body weight. According to additional embodiments, the terpinen-4-ol is administered at an amount of from 1.0 mg/Kg body weight to 10 mg/Kg body weight.

According to certain embodiments, the at least one additional anti-cancer agent is administered at the therapeutic amount known to be used for treating the specific type of cancer. According to other embodiments, the at least one additional anti-cancer agent is administered in an amount lower than the therapeutic amount known to be used for treating the disease.

According to some embodiments, the cancer is gastrointestinal cancer and the at least one additional anti-cancer agent is oxaliplatin (Eloxatin®) administered at a concentration range of from about 0.05 μM to 10 μM; or fluorouracil (5-FU) administered at a concentration range of from about 0.075 μM to about 10 μM; or cetuximab (Erbitux®) administered at a concentration range of from about 0.125 μM to 20 μM.

According to other embodiments, the cancer is pancreatic cancer, and the at least one additional anti-cancer agent is gemcitabine (Gemzar®) administered at a concentration range of from about 0.025 μM to 100 mM; or erlotinib hydrochloride (Tarceva®) administered at a concentration range of from about 0.0125 μM to 100 mM; or humanized anti-CD24 monoclonal antibodies at a concentration range of from about 20 μg/ml-2 mg/ml

According to yet additional embodiments, the cancer is prostate cancer and the at least one additional anti-cancer agent is cetuximab (Erbitux®) administered at a concentration range of from about 0.125 μM to 100 mM; or humanized anti-CD24 monoclonal antibody at a concentration range of from about 20 μg/ml-2 mg/ml.

The present invention further contemplates a method for suppressing tumor growth in a subject in need thereof, comprising administering to the subject (a) an effective amount of terpinen-4-ol and (b) an effective amount of at least one additional anti-cancer agent to provide a combination therapy having an enhanced tumor suppressing effect compared to the effect of the terpinen-4-ol and the at least one additional anti-cancer agent each administered alone.

The present invention also contemplates a method for inhibiting cancer cell proliferation, comprising contacting cancer cells with therpinen-4-ol in combination with at least one additional anti-cancer agent, wherein the combination provides an enhanced anti-cancerous effect compared to the effect of the terpinen-4-ol and the at least one additional anti-cancer agent each administered alone. According to certain exemplary embodiments, the combination of terpinen-4-ol and the at least one additional anti-cancer agent has a synergistic effect.

In yet other embodiments the present invention relates to the use of terpinen-4-ol in combination with at least one other anti-cancer agent, wherein the terpinen-4-ol and the at least one other anti-cancer agent together provide an enhanced therapeutic effect, preferably a synergistic therapeutic effect.

According to an additional aspect, the presented invention provides the use of an effective amount of terpinen-4-ol for the preparation of a medicament for treating cancer to be administered in combination with at least one additional anti-cancer agent, thereby enhancing the anti-cancerous effect compared to the effect of each of the medicament comprising the terpinen-4-ol and the at least one additional anti-cancer agent. According to certain embodiment, the anti-cancerous effect is synergistic.

According to certain exemplary embodiments, the medicament consists of the terpinen-4-ol as the sole active agent.

According to certain embodiments, the medicament comprising or consisting of the terpinen-4-ol is to be administered simultaneously with the at least one additional anti-cancer agent. According to other embodiments, the medicament comprising or consisting of the terpinen-4-ol and the at least one additional anti-cancer agent are to be administered sequentially. In some embodiments, the medicament comprising or consisting of the terpinen-4-ol and the at least one additional anti-cancer agent are to be administered concurrently. In yet other embodiments, the medicament comprising or consisting of the terpinen-4-ol and the at least one additional anti-cancer agent are to be administered non-concurrently. According to yet additional embodiments, the terpinen-4-ol and the at least one additional anti-cancer agent are to be administered in the same medicament.

According to yet additional aspect, the present invention provides a composition for treating cancer, the composition comprising a first component consisting of an effective amount of terpinen-4-ol and a second component comprising an effective amount of at least one additional anti-cancer agent. The collective amount of terpinen-4-ol and the at least one additional anti-cancer agent provides for an enhanced therapeutic anti-cancer effect. According to certain embodiments, the collective amount of terpinen-4-ol and the at least one additional anti-cancer agent provides for a synergistic therapeutic anti-cancer effect. According to certain exemplary embodiments, the composition further comprises a pharmaceutically acceptable diluents or carrier.

According to certain embodiments, the composition comprises terpinen-4-ol at a concentration range of from about 0.01% to about 99% (v/v) relative to the total volume of the composition. According to certain exemplary embodiments, the concentration of terpinen-4-ol is from about 0.1% to 80% or from 0.1% to 70% (v/v) relative to the total volume of the composition.

According to certain embodiments, the composition is administered in an amount as to provide terpinen-4-ol at an amount of from 0.1 mg/Kg body weight to 100 mg/Kg body weight. According to certain exemplary embodiments, the composition is administered in an amount as to provide terpinen-4-ol at an amount of from 0.5 mg/Kg body weight to 20 mg/Kg body or from 1.0 mg/Kg body weight to 10 mg/Kg body weight.

According to certain embodiments, the at least one additional anti-cancer agent is known to have a therapeutic effect on the cancer type to be treated.

According to certain embodiments, the cancer to be treated is gastrointestinal cancer and the composition comprises at least one additional anti-cancer agent selected from the group consisting of oxaliplatin (Eloxatin®), fluorouracil (5-FU), anti-CD24 antibody, cetuximab (Erbitux®) and bevacizumab (Avastin®). Each possibility represents a separate embodiment of the present invention.

According to other embodiments, the cancer to be treated is pancreatic cancer, and the composition comprises at least one additional anti-cancer agent selected from the group consisting of gemcitabine (Gemzar®) erlotinib hydrochlorides (Tarceva®) and humanized anti-CD24 monoclonal antibodies. Each possibility represents a separate embodiment of the present invention.

According to yet additional embodiments, the cancer to be treated is prostate cancer and the composition comprises at least one additional anti-cancer agent selected from the group consisting of cetuximab (Erbitux®), bevacizumab (Avastin®) and humanized anti-CD24 monoclonal antibodies. Each possibility represents a separate embodiment of the present invention.

According to certain exemplary embodiments, the composition comprises oxaliplatin at a concentration range of from about 0.05 μM to 10 μM. According to other embodiments, the composition comprises fluorouracil at a concentration range of from about 0.075 μM to about 10 μM. According to yet additional embodiments, the composition comprises gemcitabine at a concentration range of from about 0.025 μM to 20 μM. According to further embodiments, the composition comprises erlotinib hydrochlorides at a concentration range of from about 0.0125 μM to 20 μM. According to yet further embodiments, the composition comprises cetuximab at a concentration range of from about 0.125 μM to 20 μM. According to still further embodiments, the composition comprises humanized anti-CD24 monoclonal antibodies at a concentration range of from about 75 μg/ml-1.5 mg/ml.

According to some aspects, the present invention provides the use of an effective amount of terpinen-4-ol and an effective amount of at least one additional anti-cancer agent for the preparation of a medicament for treating cancer, wherein the collective amount of terpinen-4-ol and the at least one additional anti-cancer agent provides for an enhanced therapeutic anti-cancer effect. According to certain embodiments, the collective amount of terpinen-4-ol and the at least one additional anti-cancer agent provides for a synergistic therapeutic anti-cancer effect.

Other objects, features and advantages of the present invention will become clear from the following description and drawings.

### BRIEF DESCRIPTION OF THE FIGURES

FIG. 1 demonstrates that DMSO is not necessary for terpinen-4-ol to exert its cytotoxic activity on various cancer cell lines. DMSO at a concentration of 0.01% was added to the medium containing 0.01%, 0.05% or 0.10% terpinen-4-ol. FIG. 1A-1B: Colorectal cancer cell lines DLD1 and HT29, respectively. FIG. 1C-1E: Pancreatic cancer cell lines PANC-1, MIA-PACA and COLO 357, respectively. FIG. 1F-1G: Prostate cancer cell lines DU145 and CL-1, respectively.

FIG. 2 demonstrates the concentration-dependent activity of terpinen-4-ol. FIG. 2A-2B: Colorectal cancer cell lines DLD1 and HT29, respectively. FIG. 2C-2D: Prostate cancer cell lines DU145 and CL-1, respectively. FIG. 2E-2G: Pancreatic cancer cell lines MIA-PACA, PANC-1, and COLO 357, respectively.

FIG. 3 shows that a combination of terpinen-4-ol and at least one additional chemotherapeutic agent is more cytotoxic to cancer cells compared to the cytotoxic activity of each compound alone. Terpinen-4-ol is designated 1A. FIG. 3A-3B: effect of terpinen-4-ol and oxaliplatin or 5-FU, (respectively) on the colorectal cancer cell line DLD1. FIG. 3C-3D: effect of terpinen-4-ol and oxaliplatin or 5-FU (respectively) on the colorectal cancer cell line HT29. FIG. 3E-3G: effect of terpinen-4-ol and gemcitabine or a combination of gemcitabine and Tarceva® (erlotinib hydrochlorides) (respectively) on the pancreatic cancer cell line MIA-PACA. FIG. 3H-3I: effect of terpinen-4-ol and gemcitabine or a combination of gemcitabine and Tarceva® (erlotinib hydrochlorides) (respectively) on the pancreatic cancer cell line PANC-1. FIG. 3J: effect of terpinen-4-ol and a combination of gemcitabine and Tarceva® (erlotinib hydrochlorides) (respectively) on the pancreatic cancer cell line COLO 357. Abbreviations: A1—terpinen-4-ol; Oxal—oxaliplatin; G—gemcitabine; T—Tarceva®.

FIG. 4 shows that a combination of terpinen-4-ol and an anti-cancer antibody is more cytotoxic to cancer cells compared to the cytotoxic activity of each compound alone. FIG. 4A-4C: effect of terpinen-4-ol and humanized anti-CD24 antibodies, Erbitux® (cetuximab) or Avastin® (bevacizumab) (respectively) on the colorectal cancer cell line DLD1. FIG. 4D: effect of terpinen-4-ol and humanized anti-CD24 antibodies on the colorectal cancer cell line HT29. FIG. 4E: effect of terpinen-4-ol and humanized anti-CD24 antibodies on the pancreatic cell line PANC-1. FIG. 4F: effect of terpinen-4-ol and humanized anti-CD24 antibodies on the pancreatic cell line COLO 357. FIG. 4G-I: effect of terpinen-4-ol and humanized anti-CD24 antibodies, Erbitux® (cetuximab) or Avastin® (bevacizumab) (respectively) on the prostate cancer cell line CL-1. Abbreviations: A1—terpinen-4-ol; Ab: humanized anti-CD24 antibodies

FIG. 5 shows the effect of terpinen-4-ol on tumor development in a xenograft model of nude mice bearing DLD-1 colorectal cancer cell lines. FIG. 5A shows the tumor volume at various time points during the assay and FIG. 5B shows the weight of tumors at the end of the assay in mice treated with terpinen-4-ol compared to control mice.

FIG. 6 shows the effect of a combination therapy of terpinen-4-ol and cetuximab (Erbitux®) on tumor development in a xenograft model of nude mice bearing DLD-1 colorectal cancer cell lines. Volume measures were taken at the specified dates, before injection of the sequential treatment with terpinen-4-ol and cetuximab. Abbreviations: A1—terpinen-4-ol Erb: Erbitux® (cetuximab).

### DETAILED DESCRIPTION OF THE INVENTION

The present invention relates to the use of a combination of plant-derived anti-cancer substances and chemotherapeutic or biological agents for treating cancer. The combination therapy of the invention is highly effective in treating various types of cancer and shows enhanced effect compared to the activity of each of the components administered alone or to the additive activities of each compound (synergistic effect).

### DEFINITIONS

The term “terpinen-4-ol” is used herein according to its meaning as is known in the art. This monoterpene is one of the principal components of the tea tree oil and is also present in other plant species. Synonyms include 1-isopropyl-4-methyl-cyclohex-3-enol; 1-Methyl-4-isopropyl-1-cyclohexen-4-ol; 1-methyl-4-isopropyl-1-cyclohexen-4-ol (4-terpineol); 1-para-Menthen-4-ol; 4-methyl-1-(1-methylethyl)-3-cyclohexen-1-ol; 4-methyl-1-(1-methylethyl)-3-Cyclohexen-1-01; 4-Methyl-1-(methylethyl)-3-cyclohexen-1-ol; 4-methyl-1-isopropyl-3-cyclohexen-1-ol. The term “terpinen-4-ol” comprises all its isomers, geometrical isomers, enantiomers, diastereomers and pharmaceutically salts thereof.

As used herein, the term “cancer” includes all cancers and cancer metastases, including sarcomas, carcinomas and other solid and non-solid tumor cancers. Solids cancer include but are not limited to tumors of the central nervous system, breast cancer, prostate cancer, skin cancer (including basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, rhabdomyosarcomas, Wilms tumors, and the like. According to certain embodiments of the present invention, the cancer is selected from the group consisting of cancers of the gastrointestinal tract, pancreatic cancer and prostate cancer. Each possibility represents a separate embodiment of the present invention. According to some embodiments, the cancer of the gastrointestinal tract is selected from the group consisting of colorectal cancer and gastric cancer. According to certain embodiments, the term “cancer” further comprises pre-cancerous lesions.

The term “subject” as used herein refers to any mammal having cancer which requires treatment. Typically, the mammal is human; however, it should be explicitly understood that the mammal can also be a companion animal, for example a dog or a cat.

The terms “treating”, “treatment” and the like are used herein to mean affecting a subject, tissue or cell to obtain a desired pharmacological and/or physiological effect. The effect may be therapeutic in terms of a partial or complete cure of the cancer. “Treating” as used herein covers any treatment of cancer in a subject; inhibiting the cancer, i.e. arresting its development; or relieving or ameliorating the effects of the cancer, i.e., cause regression of the tumor or of the effects of the cancer.

The term “anti-cancer” as used herein in reference to “anti-cancer agent”, “anti-cancer therapeutic effect” “anti-cancerous effect” and the like is meant in its broadest scope as in known in the art, and includes the activities of arrest of cell growth, induction of apoptosis, induction of differentiation, cell death and the like.

As used herein, the terms “effective amount” refers to an amount of terpinen-4-ol or another anti-cancer agent according to the teachings of the present invention that is effective in treating cancer as defined hereinabove. The specific “effective amount” will vary according to the particular condition being treated, the physical condition and clinical history of the subject, the duration of the treatment and the nature of the combination of agents applied and its specific formulation. As used herein, the term “therapeutically effective amount” refers to the amount of terpinen-4-ol and/or the at least one additional anti-cancer agent known in the art to be effective in treating cancer cells/disease of a particular type. According to certain embodiments, the “effective amount” according to the teachings of the present invention is lower compared to the “therapeutically effective amount” as is known in the art.

The term “enhanced effect” and its various grammatical variations is used herein to refer to an interaction between terpinen-4-ol and at least one other agent wherein the observed effect (e.g., cytotoxicity) in the presence of the drugs together is significantly higher than the effect of each individual drug (e.g., cytotoxicities) administered separately. In one embodiment, the observed combined effect of the drugs is significantly higher than each of the individual effects. In certain embodiments the term significant means that the observed p<0.05.

The term “synergistic” and its various grammatical variations is used herein to refer to an interaction between terpinen-4-ol and at least one other agent wherein the observed effect (e.g., cytotoxicity) in the presence of the drugs together is higher than the sum of the individual effects (e.g., cytotoxicities) of each drug administered separately. In one embodiment, the observed combined effect of the drugs is significantly higher than the sum of the individual effects. In certain embodiments the term significant means that the observed p<0.05.

As used herein, the term “oxaliplatin” (trademarked as Eloxatin®) refers to an antineoplastic agent with the molecular formula C₈H₁₄N₂O₄Pt and the chemical name of cis-[(1R,2R)-1,2-cyclohexanediamine-N,N′][oxalato(2)-O,O′]platinum. Oxaliplatin is an organo-platinum complex in which the platinum atom is complexed with 1,2-diaminocyclohexane (DACH) and with an oxalate ligand as a leaving group.

The terms “fluorouracil” or “5-FU” as used herein refer to pyrimidine analog that inhibits thymidylate synthase, leading to inhibition of DNA and RNA synthesis and to cell death.

As used herein, the term gemcitabine (trademarked as Gemzar®) refers to a chemotherapeutic drug containing the active ingredient, gemcitabine hydrochloride (Chemical Formula C₉H₁₁F₂N₃O₄HCl) and the inactive ingredients mannitol and sodium acetate. It is a nucleoside analog and classified as an antimetabolite.

As used herein the term erlotinib hydrochlorides (trademarked as Tarceva®) refers to a reversible tyrosine kinase inhibitor, which acts on the epidermal growth factor receptor (EGFR).

The term “cetuximab” (trademarked as Erbitux®) refers to an immunotherapy drug used for treating head and neck cancer as well as certain types of colorectal cancer, that contains the active ingredient cetuximab, a humanized monoclonal antibody. Cetuximab specifically recognizes and binds to the epidermal growth factor receptor (EGFR) protein on the surface of cancer cells. This binding blocks the receptor, stopping growth factors from attaching to the receptor and stimulates the cells growth and multiplication. It also triggers the immune system to attack the cancerous cells.

The term “bevacizumab” (trademarked as Avastin®) as used herein refers to a recombinant humanized monoclonal IgG1 antibody that binds to and inhibits the biologic activity of human vascular endothelial growth factor (VEGF). Bevacizumab contains human framework regions and the complementarity-determining regions of a murine antibody that binds to VEGF. Avastin® has an approximate molecular weight of 149 kD.

As used herein, the term “humanized Anti-CD24 monoclonal antibody” refers to a non-matured humanized IgG1 antibody that binds to the cell surface CD24 protein. It is a non-commercial antibody that was produced from the murine SWA11 monoclonal antibodies.

According to one aspect, the present invention provides a method for treating cancer, the method comprises administering to a subject in need thereof (a) an effective amount of terpinen-4-ol and (b) an effective amount of at least one anti-cancer agent to provide a combination therapy having an enhanced anti-cancerous effect compared to the effect of the terpinen-4-ol and the at least one additional anti-cancer agent each administered alone. According to certain exemplary embodiments, the combination therapy has a synergistic therapeutic effect.

The terpinen-4-ol can be synthetically produced or it can be isolated form tea tree oil and other plants by methods well known to a person skilled in the art. According to certain typical embodiments, terpinen-4-ol is isolated from tea tree oil.

Reference to a chemotherapeutic agent herein applies to the chemotherapeutic agent or its derivatives and accordingly the invention contemplates and includes either of these embodiments (agent; agent or derivative(s)). “Derivatives” or “analogs” of a chemotherapeutic agent or other chemical moiety include, but are not limited to, compounds that are structurally similar to the chemotherapeutic agent or moiety or are in the same general chemical class as the chemotherapeutic agent or moiety. The derivative or analog of the chemotherapeutic agent or moiety retains similar chemical and/or physical property (including, for example, functionality) of the chemotherapeutic agent or moiety.

Suitable chemotherapeutic agents for use in the combinations of the present invention include, but are not limited to, alkylating agents, antibiotic agents, antimetabolic agents, hormonal agents, plant-derived agents, anti-angiogenic agents, differentiation inducing agents, cell growth arrest inducing agents, apoptosis inducing agents, cytotoxic agents, agents affecting cell bioenergetics, biologic agents, e.g., monoclonal antibodies, kinase inhibitors and inhibitors of growth factors and their receptors, gene therapy agents, cell therapy, e.g., stem cells, or any combination thereof.

Alkylating agents are drugs which impair cell function by forming covalent bonds with amino, carboxyl, sulfhydryl and phosphate groups in biologically important molecules. The most important sites of alkylation are DNA, RNA and proteins. Alkylating agents depend on cell proliferation for activity but are not cell-cycle-phase-specific. Alkylating agents suitable for use in the present invention include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitroso-ureas (e. g. BCNU, carmustine, lomustine, streptozocin), nonclassic alkylating agents (e.g., altretamine, dacarbazine, and procarbazine), and platinum compounds (e.g., carboplastin, oxaliplatin and cisplatin).

Antitumor antibiotics like adriamycin intercalate DNA at guanine-cytosine and guanine-thymine sequences, resulting in spontaneous oxidation and formation of free oxygen radicals that cause strand breakage. Other antibiotic agents suitable for use in the present invention include, but are not limited to, anthracyclines (e. g. doxorubicin, daunorubicin, epirubicin, idarubicin and anthracenedione), mitomycin C, bleomycin, dactinomycin, and plicatomycin.

Antimetabolic agents suitable for use in the present invention include but are not limited to, floxuridine, fluorouracil, methotrexate, leucovorin, hydroxyurea, thioguanine, mercaptopurine, cytarabine, pentostatin, fludarabine phosphate, cladribine, asparaginase, and gemcitabine.

Hormonal agents suitable for use in the present invention, include but are not limited to, an estrogen, a progestogen, an antiesterogen, an androgen, an antiandrogen, an LHRH analogue, an aromatase inhibitor, diethylstibestrol, tamoxifen, toremifene, fluoxymesterol, raloxifene, bicalutamide, nilutamide, flutamide, aminoglutethimide, tetrazole, ketoconazole, goserelin acetate, leuprolide, megestrol acetate, and mifepristone.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis. Other plant derived agents include, but are not limited to, vincristine, vinblastine, vindesine, vinzolidine, vinorelbine, etoposide, teniposide, and docetaxel.

Biologic agents suitable for use in the present invention include, but are not limited to immuno-modulating proteins, monoclonal antibodies against tumor antigens, tumor suppressor genes, kinase inhibitors and inhibitors of growth factors and their receptors and cancer vaccines. For example, the immuno-modulating protein can be interleukin 2, interleukin 4, interleukin 12, interferon El interferon D, interferon alpha, erythropoietin, granulocyte-CSF, granulocyte, macrophage-CSF, bacillus Calmette-Guerin, levamisole, or octreotide. Examples of biologic agents to be used according to the teachings of the present invention are anti-CD24 antibody, cetuximab (Erbitux®) and bevacizumab (Avastin®)

Agents affecting cell bioenergetics affecting cellular ATP levels and/or molecules/activities regulating these levels

Recent developments have introduced, in addition to the traditional cytotoxic and hormonal therapies, additional therapies for the treatment of cancer. For example, many forms of gene therapy are undergoing preclinical or clinical trials. In addition, approaches based on the inhibition of tumor vascularization (angiogenesis) are currently under development. The aim of this concept is to cut off the tumor from nutrition and oxygen supply provided by a newly built tumor vascular system. In addition, cancer therapy is also being attempted by the induction of terminal differentiation of the neoplastic cells. Suitable differentiation agents include hydroxamic acids, derivatives of vitamin D and retinoic acid, steroid hormones, growth factors, tumor promoters, and inhibitors of DNA or RNA synthesis. Also, histone deacetylase inhibitors are suitable chemotherapeutic agent to be used in the present invention.

According to certain embodiments, the at least one additional anti-cancer agent is known to be effective in treating the cancer type affecting the subject.

According to some embodiments, the method is for treating gastrointestinal cancer. According to certain exemplary embodiments, the method comprises administering to the subject an effective amount of terpinen-4-ol and an effective amount of oxaliplatin (Eloxatin®), thereby treating gastrointestinal cancer. According to other exemplary embodiments, the method comprises administering to the subject an effective amount of terpinen-4-ol and an effective amount of fluorouracil (5-FU), thereby treating gastrointestinal cancer. According to additional exemplary embodiments, the method comprises administering to the subject an effective amount of terpinen-4-ol and an effective amount of anti-CD24 antibody, thereby treating gastrointestinal cancer. According to yet further exemplary embodiments, the method comprises administering to the subject an effective amount of terpinen-4-ol and an effective amount of cetuximab (Erbitux®), thereby treating gastrointestinal cancer. According to yet additional exemplary embodiments, the method comprises administering to the subject an effective amount of terpinen-4-ol and an effective amount of and bevacizumab (Avastin®) thereby treating gastrointestinal cancer. According to certain embodiments, the gastrointestinal cancer is selected from the group consisting of gastric cancer and colorectal cancer.

According to other embodiments, the method is for treating pancreatic cancer. According to certain exemplary embodiments, the method comprises administering to the subject an effective amount of terpinen-4-ol and an effective amount of gemcitabine (Gemzar®), thereby treating pancreatic cancer. According to other exemplary embodiments, the method comprises administering to the subject an effective amount of terpinen-4-ol and an effective amount of erlotinib hydrochlorides (Tarceva®), thereby treating pancreatic cancer. According to additional exemplary embodiments, the method comprises administering to the subject an effective amount of terpinen-4-ol and an effective amount of humanized anti-CD24 monoclonal antibodies, thereby treating pancreatic cancer.

According to yet additional embodiments, the method is for treating prostate cancer. According to certain exemplary embodiments, the method comprises administering to the subject an effective amount of terpinen-4-ol and an effective amount of cetuximab (Erbitux®), thereby treating prostate cancer. According to additional exemplary embodiments, the method comprises administering to the subject an effective amount of terpinen-4-ol and an effective amount of bevacizumab (Avastin®), thereby treating prostate cancer. According to other exemplary embodiments, the method comprises administering to the subject an effective amount of terpinen-4-ol and an effective amount of humanized anti-CD24 monoclonal antibodies, thereby treating prostate cancer.

Determining the dosage and duration of treatment according to any aspect of the present invention is well within the skills of a professional in the art. The skilled Artisans are readily able to monitor patients to determine whether treatment should be started, continued, discontinued or resumed at any given time. For example, dosages of the compounds are suitably determined depending on the individual cases taking symptoms, age and sex of the subject and the like into consideration. The amount of the compound to be incorporated into the pharmaceutical composition of the invention varies with dosage route, solubility of the compound, administration route, administration scheme and the like. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum effect is achieved. Suitable dosages can be determined by further taking into account relevant disclosure in the known art.

According to certain embodiments, the terpinen-4-ol is administered in an amount sufficient so as to allow reduction of the normal dose of the at least one additional anti-cancer agent required to effect the same degree of treatment by at least about any of 5%, 10%, 20%, 30%, 50%, 60%, 70%, 80%, 90%, or more. According to other embodiments, the at least one additional anti-cancer agent is administered in an amount sufficient so as to allow reduction of the normal dose of the chemotherapeutic agent required to effect the same degree of treatment by at least about any of 5%, 10%, 20%, 30%, 50%, 60%, 70%, 80%, 90%, or more.

According to certain embodiments, the terpinen-4-ol is administered in an amount of from 0.1 mg/Kg body weight to 100 mg/Kg body weight. According to other embodiments, the terpinen-4-ol is administered at an amount of from 0.5 mg/Kg body weight to 20 mg/Kg body weight. According to additional embodiments, the terpinen-4-ol is administered at an amount of from 1.0 mg/Kg body weight to 10 mg/Kg body weight.

However, it is to be explicitly understood that lower or higher concentrations of terpinen-4-ol can be used, depending on the additional anti-cancer agent(s) present in the composition, the subject to be treated (age, gender, weight etc.), the type of cancer to be treated and the stage of the disease. As exemplified hereinbelow, the combination of terpinen-4-ol and at least one additional anti-cancer agent significantly reduced the survival of cancer cell lines. The inhibiting activity of the combination was significantly higher as compared to the additive inhibition activity of each of the component, and thus defined as synergistic effect.

The terpinen-4-ol or a composition comprising same (“the terpinen-4-ol”) and the at least one additional anti-cancer agent or a composition comprising same (“additional anti-cancer agent”) can be administered simultaneously (i.e., simultaneous administration) and/or sequentially (i.e., sequential administration).

According to some embodiments, the terpinen-4-ol and the at least additional anti-cancer agent are administered simultaneously. The term “simultaneous administration,” as used herein, means that the terpinen-4-ol and the at least additional anti-cancer agent are administered with a time separation of no more than about 15 minute(s), such as no more than about any of 10, 5, or 1 minutes. When the drugs are administered simultaneously, the terpinen-4-ol and the at least additional anti-cancer agent may be contained in the same composition (e.g., a composition comprising both the terpinen-4-ol and the at least additional anti-cancer agent) or in separate compositions (e.g., the terpone-4-ol is contained in one composition and the at least additional anti-cancer agent is contained in another composition).

According to other embodiments, the terpinen-4-ol and the at least one additional anti-cancer agent are administered sequentially. The term “sequential administration” as used herein means that the terpinen-4-ol and the additional anti-cancer agent are administered with a time separation of more than about 15 minutes, such as more than about any of 20, 30, 40, 50, 60 or more minutes. Either the terpinen-4-ol or the additional anti-cancer agent may be administered first. The terpinen-4-ol and the additional anti-cancer agent are contained in separate compositions, which may be contained in the same or different packages.

According to yet additional embodiments, the administration of the terpinen-4-ol and the at least one additional anti-cancer agent are concurrent, i.e., the administration period of the terpinen-4-ol and that of the at least one additional anti-cancer agent overlap with each other. In some embodiments, the administration of the terpinen-4-ol and the at least one additional anti-cancer agent are non-concurrent. For example, in some embodiments, the administration of the terpinen-4-ol is terminated before the at least one additional anti-cancer agent is administered. In some embodiments, the administration of the at least one additional anti-cancer agent is terminated before the terpinen-4-ol is administered. The time period between these two non-concurrent administrations can range from being days apart to being weeks apart.

The dosing frequency of the terpinen-4-ol and the at least one additional anti-cancer agent may be adjusted over the course of the treatment, based on the judgment of the administering physician. When administered separately, the terpinen-4-ol and the at least one additional anti-cancer agent can be administered at different dosing frequency or intervals. For example, the terpinen-4-ol can be administered weekly, while the at least one additional anti-cancer agent can be administered more or less frequently. In some embodiments, sustained continuous release formulation of both components may be used. Various formulations and devices for achieving sustained release are known in the art. In addition, the terpinen-4-ol and the at least one additional anti-cancer agent can be administered using the same route of administration or using different routes of administration.

It is to be explicitly understood that the present invention further encompasses combinations of the various administration configurations described herein. The methods described herein employing a combination of terpinen-4-ol and at least one additional anti-cancer agent can be performed alone or in combination with another therapy, including surgery, radiation, chemotherapy, immunotherapy, gene therapy, and the like.

Although the components of the combination therapy of the present invention can be administered alone, it is contemplated that the components of the combination will be administered in pharmaceutical compositions further containing at least one pharmaceutically acceptable carrier or excipient. Each of the components can be administered in a separate pharmaceutical composition, or the combination can be administered in one pharmaceutical composition.

Thus, according to additional aspect, the present invention provides a composition for treating cancer, the composition comprising a first component consisting of an effective amount of terpinen-4-ol and a second component comprising an effective amount of at least one additional anti-cancer agent. The terpinen-4-ol and the at least one other anti-cancer agent together provide a therapeutic anti-cancer effect which is at least enhanced compared to the effect of each of the components administered alone, and, in one embodiment, is synergistic.

According to certain exemplary embodiments, the compositions of the present invention are pharmaceutical compositions further comprising pharmaceutically acceptable diluents, excipients or carriers.

According to certain embodiments, the pharmaceutical composition comprises terpinen-4-ol at a concentration range of from about 0.01% to about 99% (v/v) relative to the total volume of the composition. According to certain exemplary embodiments, the concentration of terpinen-4-ol is from about 0.1% to 90% or from 0.1% to 80% or from 0.1% to 70% (v/v) relative to the total volume of the composition.

According to certain embodiments, the pharmaceutical composition is administered in an amount as to provide terpinen-4-ol at an amount of from 0.01 mg/Kg body weight to 100 mg/Kg body weight. According to certain exemplary embodiments, the composition is administered in an amount as to provide terpinen-4-ol at an amount of from 0.5 mg/Kg body weight to 20 mg/Kg body or from 1.0 mg/Kg body weight to 10 mg/Kg body weight.

The pharmaceutical compositions of the present invention can be formulated for administration by a variety of routes. The pharmaceutical compositions may be administered orally, parenterally, by inhalation spray, topically, rectally, nasally, buccally, vaginally or via an implanted reservoir. Direct administration to solid tumor is explicitly encompassed by the present invention. The pharmaceutical compositions may contain any conventional non-toxic pharmaceutically acceptable carriers, adjuvants or vehicles. Parenteral administration of the compositions may include subcutaneous, intracutaneous, intravenous, intramuscular, intraperitoneal, intraarticular, intrasynovial, intrasternal, intrathecal, intralesional and intracranial injection or infusion techniques. Typically, administration rout would be adapted according to the type of the cancer to be treated and the formulae of the compositions of the invention.

The pharmaceutical compositions of the invention can be administered locally or systemically. By systemic administration means any mode or route of administration that results in effective amounts of the active ingredients appearing in the blood or at a site remote from the route of administration of the active ingredients.

Methods for the preparation of pharmaceutical and therapeutic compositions are well known in the art, as described, for example, in Remington's Pharmaceutical Sciences, 18th Edition, Mack Publishing Company, Easton, Pa., USA.

During the preparation of the pharmaceutical compositions according to the present invention the active ingredient is usually mixed with a carrier or excipient, which may be a solid, semi-solid, or liquid material. The compositions can be in the form of tablets, pills, capsules, pellets, granules, powders, lozenges, sachets, cachets, elixirs, suspensions, dispersions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfate), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

In one embodiment, in the pharmaceutical composition the active ingredient is dissolved in any acceptable lipid carrier (e.g., fatty acids, oils to form, for example, a micelle or a liposome).

For preparing solid compositions such as tablets, the principal active ingredient(s) is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing the desired amount of the active compounds.

Any method can be used to prepare the pharmaceutical compositions. Solid dosage forms can be prepared by wet granulation, dry granulation, direct compression and the like. The solid dosage forms of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art.

In yet another embodiment, the composition is prepared for topical administration, e.g. as an ointment, a gel a drop or a cream. For topical administration to body surfaces using, for example, creams, gels, drops, ointments and the like, the compounds of the present invention can be prepared and applied in a physiologically acceptable diluent with or without a pharmaceutical carrier. The present invention may be used topically or transdermally to treat cancer, for example, melanoma. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, pumps delivering the drugs into the body (including mechanical or osmotic pumps) controlled-release formulations and the like, as are known in the art.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

It may be desirable to administer the pharmaceutical composition of the invention locally to the area in need of treatment; this may be achieved by, for example, and not by way of limitation, local infusion during surgery, infusion to the liver via feeding blood vessels with or without surgery, topical application, e.g., in conjunction with a wound dressing after surgery, by injection, by means of a catheter, by means of a suppository, or by means of an implant, said implant being of a porous, non-porous, or gelatinous material. According to some preferred embodiments, administration can be by direct injection e.g., via a syringe, at the site of a tumor or neoplastic or pre-neoplastic tissue.

The compounds may also be administered by any convenient route, for example by infusion or bolus injection, by absorption through epithelial linings (e.g., oral mucosa, rectal and intestinal mucosa, etc.), and may be administered together with other therapeutically active agents. It is preferred that administration is localized, but it may be systemic. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer, and formulation with an aerosolizing agent.

A compound of the present invention can be delivered in an immediate release or in a controlled release system. In one embodiment, an infusion pump may be used to administer a compound of the invention, such as one that is used for delivering chemotherapy to specific organs or tumors (see Buchwald et al., 1980, Surgery 88: 507; Saudek et al., 1989, N. Engl. J. Med. 321: 574). In a preferred form, a compound of the invention is administered in combination with a biodegradable, biocompatible polymeric implant, which releases the compound over a controlled period of time at a selected site. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

Furthermore, at times, the pharmaceutical compositions may be formulated for parenteral administration (subcutaneous, intravenous, intraarterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. The above formulations may also be used for direct intra-tumoral injection. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art.

Alternatively, the combinations of the present invention can be used in hemodialysis such as leukophoresis and other related methods, e.g., blood is drawn from the patient by a variety of methods such as dialysis through a column/hollow fiber membrane, cartridge etc, is treated with the terpinen-4-ol and the chemotherapeutic drug Ex-vivo, and returned to the patient following treatment. Such treatment methods are well known and described in the art. See, e.g., Kolho et al. (J. Med. Virol. 1993, 40(4): 318-21); Ting et at (Transplantation, 1978, 25(1): 31-3); the contents of which are hereby incorporated by reference in their entirety.

According to some embodiments, the composition of the present invention is for treating gastrointestinal cancer and the at least one additional anti-cancer agent is selected from the group consisting of oxaliplatin (Eloxatin®), fluorouracil (5-FU), anti-CD24 antibody, cetuximab (Erbitux®) and bevacizumab (Avastin®).

According to other embodiments, the composition is for treating pancreatic cancer, and the at least one additional anti-cancer agent is selected from the group consisting of gemcitabine (Gemzar®) erlotinib hydrochloride (Tarceva®) and humanized anti-CD24 monoclonal antibodies.

According to yet additional embodiments, the composition is for treating prostate cancer and the at least one additional anti-cancer agent is selected from the group consisting of cetuximab (Erbitux®), bevacizumab (Avastin®) and humanized anti-CD24 monoclonal antibody.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Material and Methods

### Materials

Terpinen-4-ol: CAS No.: 562-74-3; Molecular Formula: C₁₀H₁₈O; Molecular weight: 154.25
Dimethyl sulfoxide (DMSO): CAS No. 67-68-5; Molecular Formula: (CH₃)₂SO

### Oxaliplatin (ELOXATIN®)

### Fluorouracil (5-FU)

### Gemcitabine (GEMZAR®)

Erlotinib hydrochloride (Tarceva®)

### Cetuximab (Erbitux®)

### Bevacizumab (Avastin®)

Humanized Anti-CD24 monoclonal antibody (mAb)
Cell lines used are listed in Table 1 hereinbelow.

TABLE 1
List of Examined Cell Lines

| Cell line | Cancer origin | Description | ATCC number |
|---|---|---|---|
| HCT116 | Colorectal | Human colon | CCL-247 ™ |
| | cancer | carcinoma cells, | |
| | (CRC) | colonic epithelial | |
| | | cell line | |
| HT29 | CRC | Human colon | HTB-38 ™ |
| | | adenocarcinoma | |
| | | grade II cell line | |
| COLO 320 | CRC | Human colorectal | CCL-220.1 ™ |
| | | carcinoma cell line | |
| DLD1 | CRC | colorectal adeno- | CCL-221 ™ |
| | | carcinoma cell lines | |
| AGS | Stomach | Human gastric cancer | CRL-1739 ™ |
| | | cell line | |
| COLO 357 | Pancreas | Human cell line of | |
| | | metastatic pancreatic | |
| | | adenocarcinoma | |
| PANC-1 | Pancreas | Human pancreatic | CRL-1469 |
| | | carcinoma, epithelial- | |
| | | like cell line | |
| MIA-PACA | Pancreas | Human pancreatic | CRL-1420 |
| | | carcinoma cells | |
| CL-1 | Prostate | An aggressive | |
| | | androgen-inde- | |
| | | pendent cell line | |
| DU145 | Prostate | Human prostate cell | HTB-81 |
| | | line with moderate | |
| | | metastatic potential | |

### Methods

Cell Growth Conditions

Human colon adenocarcinoma grade II cells, HT29; Human colon carcinoma cells, HCT116; Human colonic carcinoma cells, COLO 320; Human cell line of metastatic pancreatic adenocarcinoma, COLO 357; Human pancreatic carcinoma epithelial-like cell line, PANC-1; and Human pancreatic carcinoma cells, MIA-PACA were grown in high-glucose Dulbecco's modified Eagle's medium (DMEM) supplemented with 5% heat-inactivated (HI) fetal bovine serum (FBS), 1% Glutamine and streptomycin/penicillin.

Androgen-independent, highly anaplastic prostate cancer cell line with moderate metastatic potential, DU145; Aggressive androgen-independent anaplastic cell line, CL-1; and the colorectal adenocarcinoma DLD1 cells were grown in RPMI-1640 medium supplemented with 5% HI-FBS.

Cytotoxic Assay

The cytotoxic effects of terpinen-4-ol on cells were measured with the 3-[4,5-dimethylthiazol-2-yl]-2,5 diphenyltetrazolium (MTT) assay. Cells were seeded in 96-well plates (1×10⁴ cells/well) in complete medium. After 12-16 hours of growth, different concentrations of each of the described agents were added to the cells in triplicates. 72 h later, the medium was replaced with fresh medium (100 μl per well) containing 1 mg/ml MTT and the cell were further incubated for 2 to 4 h. MTT-formazan crystals were dissolved by the addition of 100 μl extraction buffer. Absorbance at 570 nm and reference wavelength of 690 nm were recorded on an automated microplate reader. The relative number of viable cells compared to control cell line grown under the same condition, but without the added examined agent was determined.

Xenograft Model for Vivo Anti-Cancer Activity

Male athymic nude mice, 6-8 weeks old, (Harlan Laboratories) were housed in sterile cages and handled with aseptic precautions. The mice were fed ad libitum. The therapeutic anti-cancer activity of terpinen-4-ol alone or in combination with additional anti-cancer agents was examined as follows: exponentially growing DLD1 cancer cells were harvested and resuspended at a final concentration of 5×10⁶ cells per 0.2 ml PBS per injection. The cells were injected subcutaneously at two sites on the backs of the mice. When tumors were palpable, mice were randomly divided into the treatment groups as described hereinbelow. Same model is used with additional types of cancer cell line as listed in Table 1 hereinabove.

Apoptosis Assay

Cells are seeded in 12-well plates (1×10⁵ cells/well) in complete medium. The different agents as described in the “cell growth conditions” section hereinabove are added for 72 h. Detection of Annexin V is performed according to the manufacturer's protocol (Annexin V Apoptosis kit, MBL). Cells are washed with PBS, and then incubated in a solution of Annexin V binding protein. Just before FACS analysis, cells are incubated in a solution of propidium iodide.

### Example 1

### The Effect of DMSO on Terpinen-4-ol Cytotoxic Activity

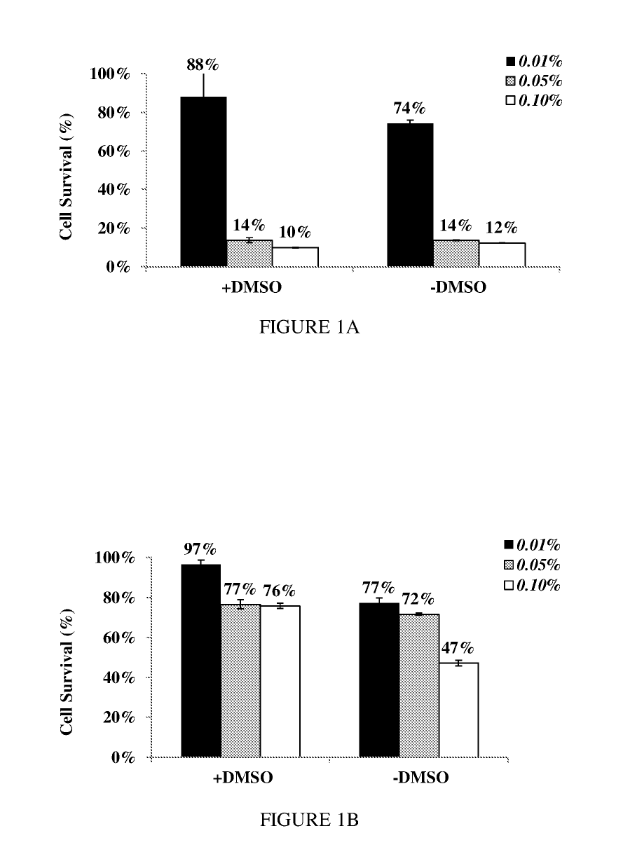

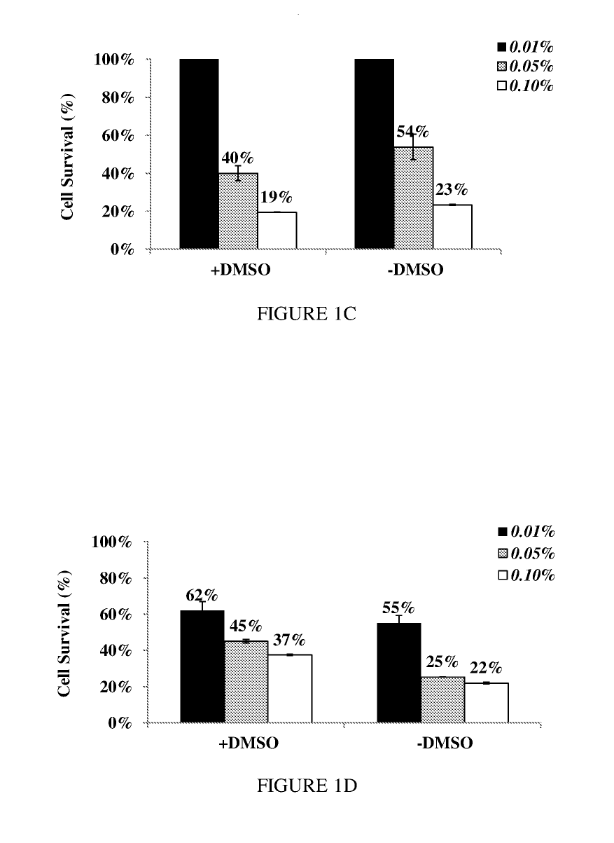

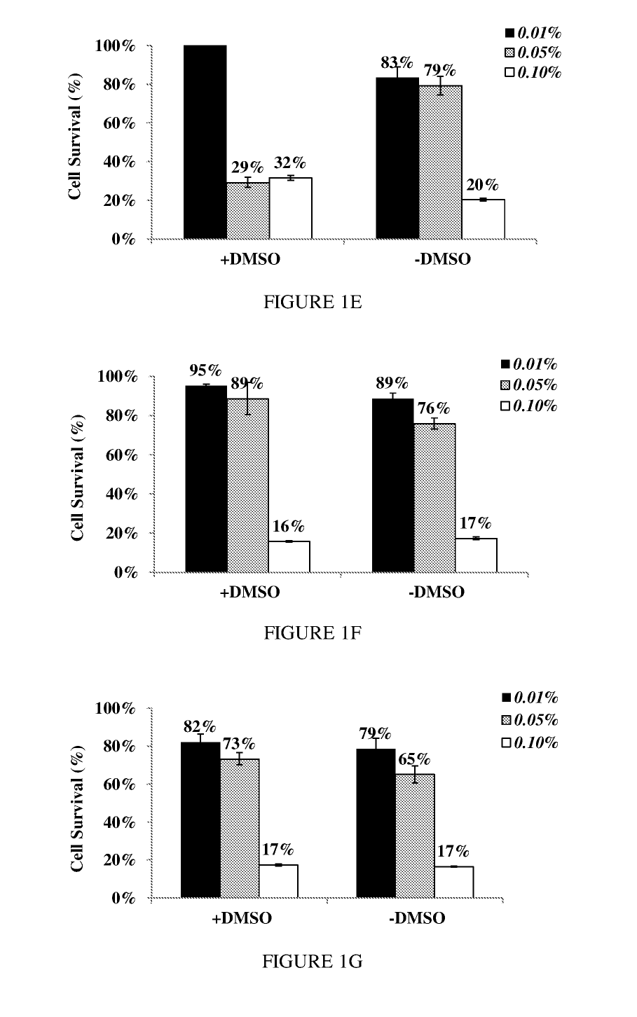

One of the obstacles in using cytotoxic compounds as therapeutics is their low solubility in pharmaceutically acceptable solutions and/or their low ability to penetrate into cells. It was therefore examined whether the addition of Dimethyl sulfoxide (DMSO) is necessary or improves the cytotoxic activity of terpinen-4-ol. As presented in FIG. 1A-1G, the cytotoxic activity of terpinen-4-ol on cancer cells of various types does not depend on the presence of DMSO; to the contrary, in several cases (e.g. with the colorectal cell line HT29, FIG. 1B), lower activity was observed in the presence of DMSO.

### Example 2

### Cytotoxic Activity of Terpinen-4-ol on Cancer Cell Lines

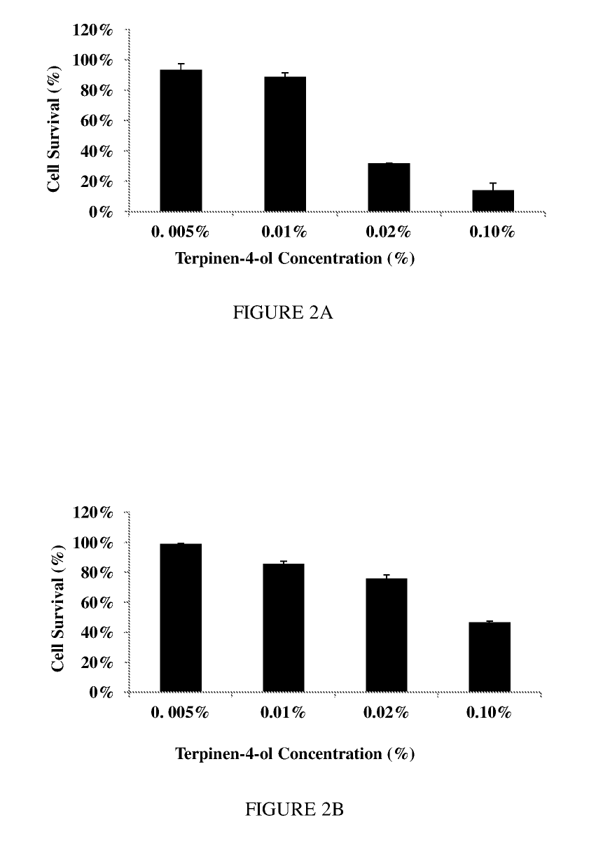

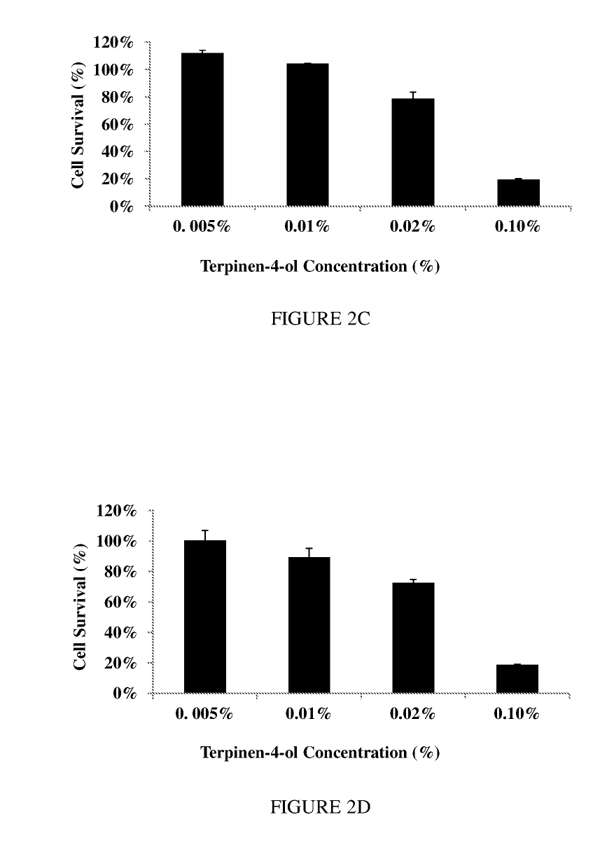

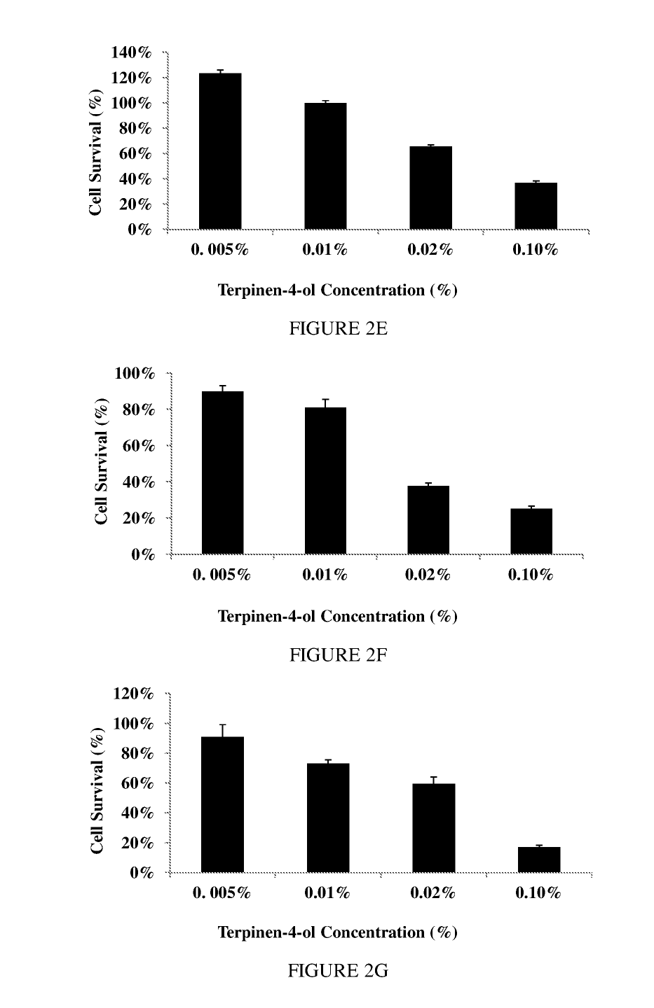

In the next step, it was examined whether the effect of terpinen-4-ol is concentration dependent. The assay conditions were as described in the “cell growth conditions” section hereinabove. Four concentrations of terpinen-4-ol were examined: 0.005%, 0.01%, 0.02% and 0.10% (v/v). As is demonstrated in FIG. 2, the cytotoxic effect of terpinen-4-ol was concentration dependent in all cancer cell lines examined.

### Example 3

### Cytotoxic Effect of a Combination of Terpinen-4-Ol and Known Chemotherapeutic Agent on Cancer Cell Lines

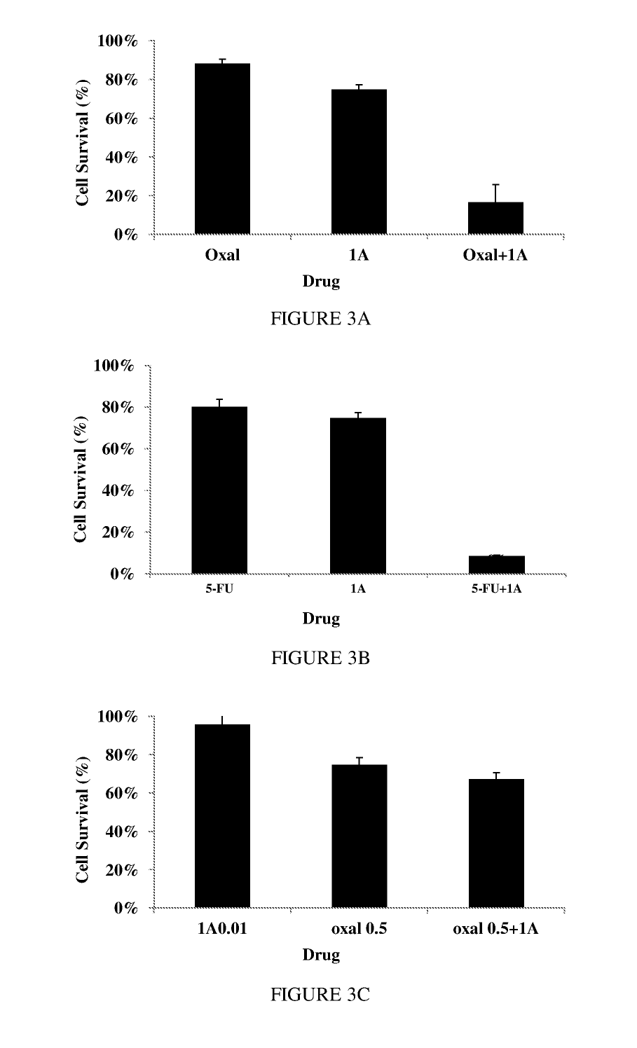

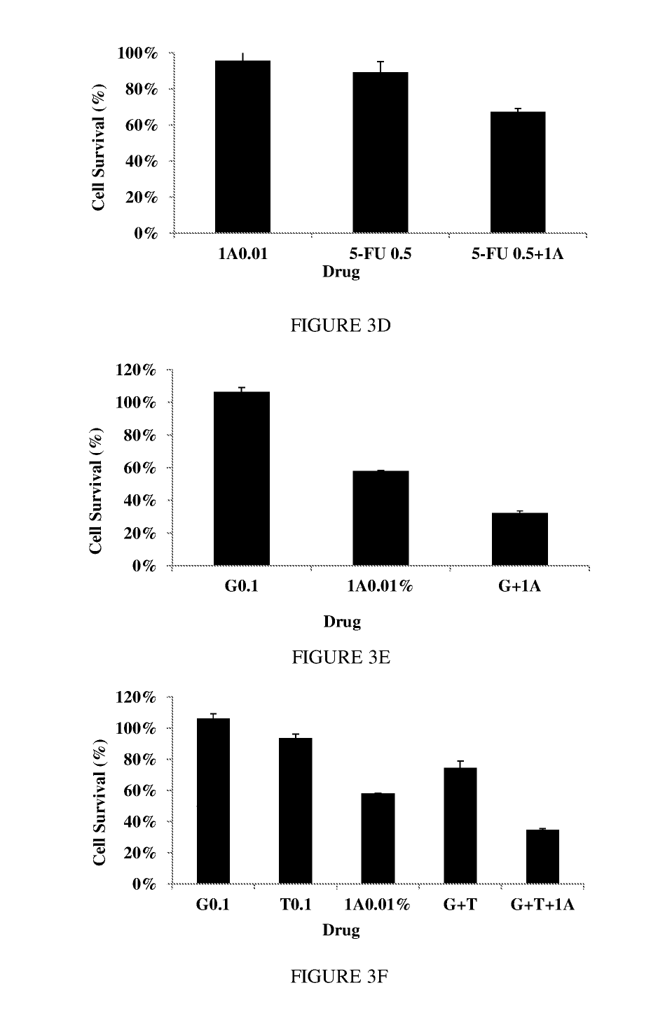

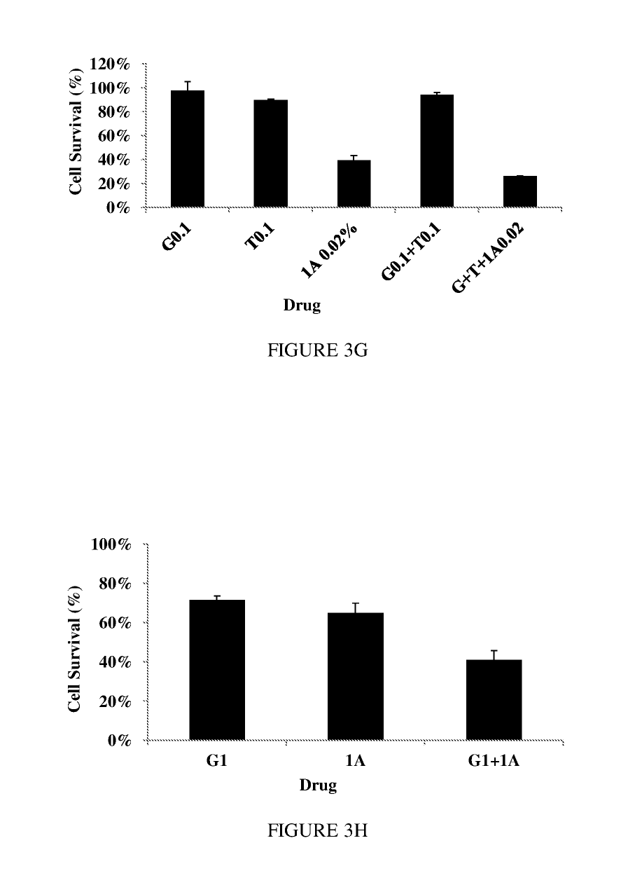

The cytotoxic effects on cancer cell line of a combination of terpinen-4-ol and different chemotherapeutic agents are demonstrated in FIG. 3. The assay conditions were as described in the “cell growth condition” section hereinabove. Terpinen-4-ol was added at a concentration of 0.01%, shown to be moderately effective, as to enable easy detection of synergistic effects. The combinations examined were as follows:

Effect on colorectal cancer cell line DLD1: Oxaliplatin at a concentration of 0.2 μM and terpinen-4-ol at a concentration of 0.01% (FIG. 3A); 5-FU at a concentration of 0.3 μM and terpinen-4-ol at a concentration of 0.01% (FIG. 3B).

Effect on colorectal cancer cell line HT29: Oxaliplatin at a concentration of 0.5 μM and terpinen-4-ol at a concentration of 0.01% (FIG. 3C); 5-FU at a concentration of 0.5 μM and terpinen-4-ol at a concentration of 0.01% (FIG. 3D).

Effect on pancreatic cancer cell line MIA-PACA: Gemcitabine at a concentration of 0.1 μM and terpinen-4-ol at a concentration of 0.01%; (FIG. 3E) Gemcitabine at a concentration of 0.1 μM; erlotinib hydrochlorides (Tarceva®) at a concentration of 0.1 μM; and terpinen-4-ol at a concentration of 0.01% (FIG. 3F); Gemcitabine at a concentration of 0.1 μM; erlotinib hydrochlorides (Tarceva®) at a concentration of 0.1 μM; and terpinen-4-ol at a concentration of 0.02% (FIG. 3G).

Effect on pancreatic cancer cell line PANC-1: Gemcitabine at a concentration of 1.0 μM and terpinen-4-ol at a concentration of 0.01% (FIG. 3H); Gemcitabine at a concentration of 1.0 μM; erlotinib hydrochlorides (Tarceva®) at a concentration of 0.1 μM; and terpinen-4-ol at a concentration of 0.01% (FIG. 3I).

Effect on pancreatic cancer cell line COLO 357: Gemcitabine at a concentration of 0.05 μM; erlotinib hydrochlorides (Tarceva®) at a concentration of 0.05 μM; and terpinen-4-ol at a concentration of 0.01% (FIG. 3J).

Chemotherapeutic agents known to have significant cytotoxic activity against the examined cancer cell line were selected in this experiment. The concentration used was also based on the effective concentration as is known in the art. In all the conditions examined, a cytotoxic synergistic effect was observed between terpinen-4-ol and the examined chemotherapeutic agent.

### Example 4

### Cytotoxic Effect of a Combination of Terpinen-4-Ol and Anti-Cancer Antibody on Cancer Cell Lines

A combination of terpinen-4-ol and several known antibodies used in the clinic as anti-cancer agents, including cetuximab (Erbitux®) and bevacizumab (Avastin®) was examined. Also examined was a humanized anti-CD24 antibody that is currently under examination (received in the courtesy of Prof. Nadir Arber). The combinations examined were as follows:

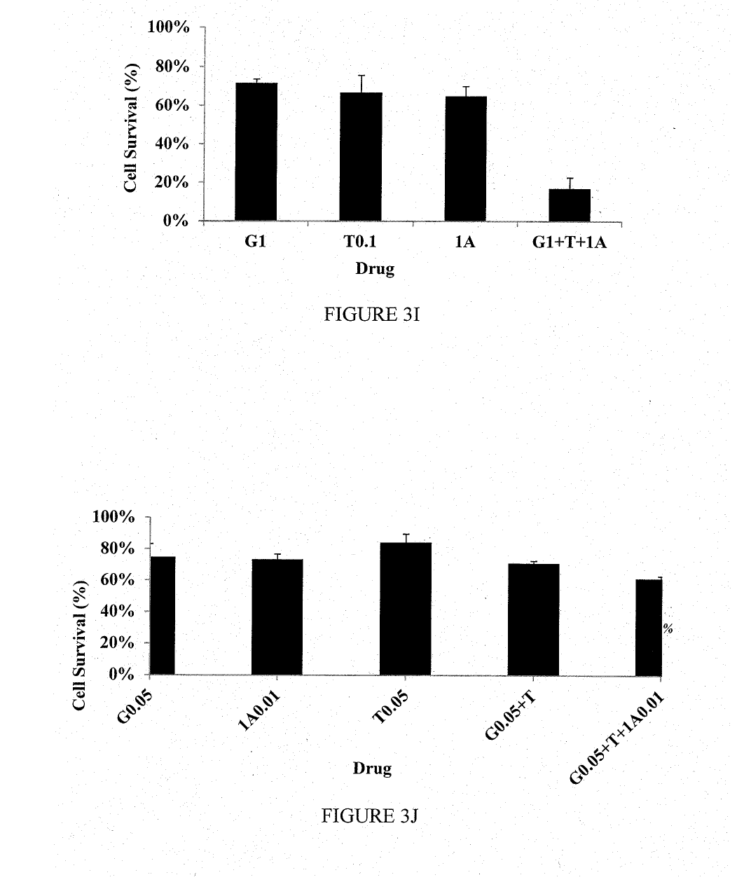

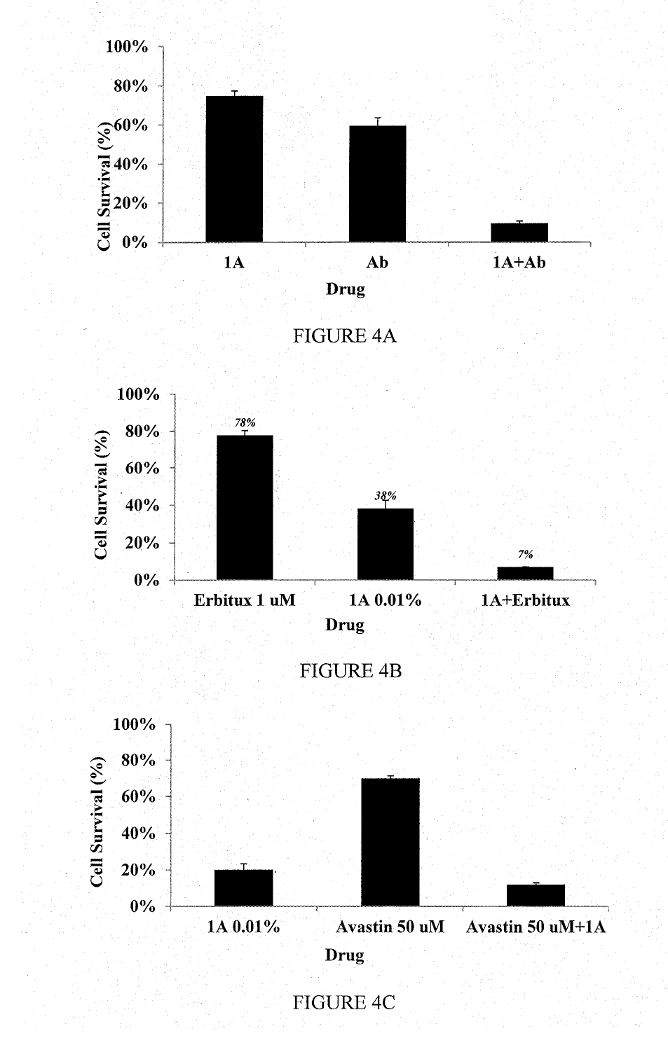

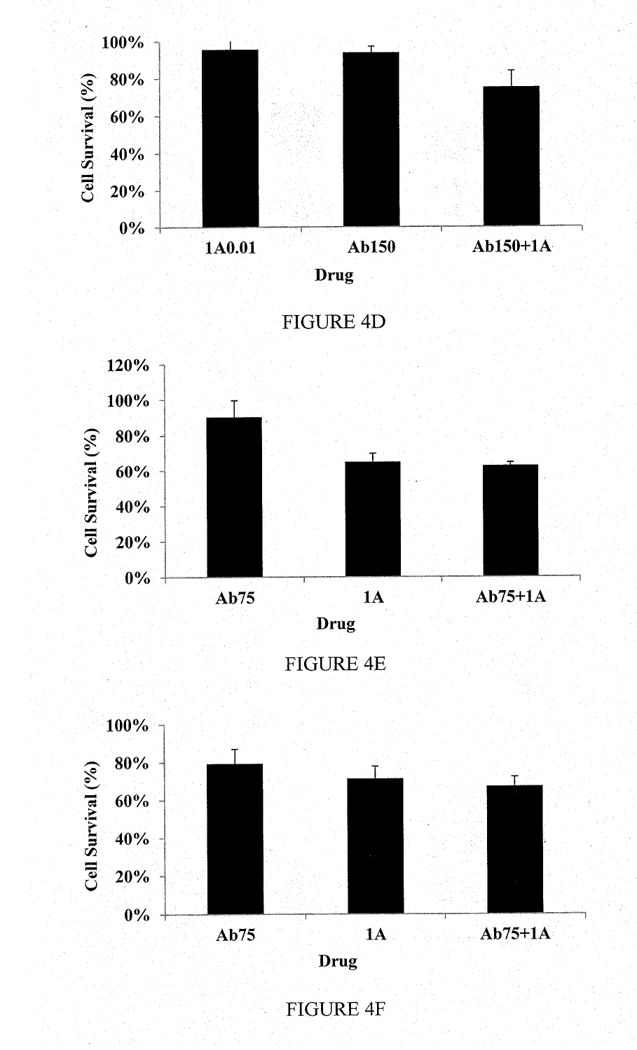

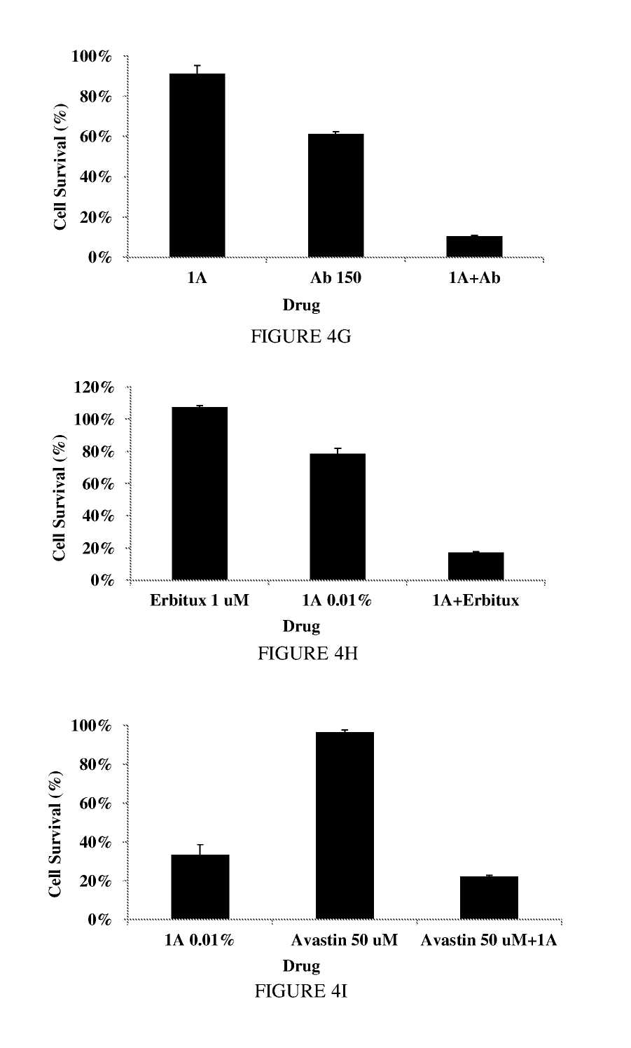

Effect on colorectal cancer cell line DLD1: terpinen-4-ol at a concentration of 0.01% and humanized anti-CD24 antibodies (150 μg/ml) (FIG. 4A); terpinen-4-ol at a concentration of 0.01% and cetuximab at a concentration of 1 μM (FIG. 4B); terpinen-4-ol at a concentration of 0.01% and bevacizumab at a concentration of 50 μM (FIG. 4C);

Effect on colorectal cancer cell line HT29: terpinen-4-ol at a concentration of 0.01% and humanized anti-CD24 antibodies (150 μg/ml) (FIG. 4D).

Effect on pancreatic cell line PANC-1: terpinen-4-ol at a concentration of 0.01% and humanized anti-CD24 antibodies (75 μg/ml) (FIG. 4E).

Effect on pancreatic cell line COLO 357: terpinen-4-ol at a concentration of 0.01% and humanized anti-CD24 antibodies (75 μg/ml) (FIG. 4F).

Effect on prostate cancer cell line CL-1: terpinen-4-ol at a concentration of 0.01% and humanized anti-CD24 antibodies (150 μg/ml) (FIG. 4G); terpinen-4-ol at a concentration of 0.01% and cetuximab at a concentration of 1 μM (FIG. 4H); terpinen-4-ol at a concentration of 0.01% and bevacizumab at a concentration of 50 μM (FIG. 4I).

A combination of terpinen-4-ol with anti-cancer antibody, that is considered to have very different mode of action compared to the chemotherapeutic agents described above, has also shown a significant synergistic cytotoxic effect against all the cancer cell lines examined.

### Example 5

### Evaluation of Terpinen-4-Ol in Xenograft Model of Nude Mice Bearing DLD-1 Colorectal Cancer Cell Line

The present study aimed to investigate the in vivo anti-cancer activity of terpinen-4-ol alone. The xenograft model using male athymic nude mice described in the method section hereinabove was used.

At the 9^{th} day after cell injection mice were randomly divided into treatment and control groups and the treatment was started by intra-tumor injection. The control group (5 mice) received 50 μl of a PBS solution with 1% DMSO; the treatment group (5 mice) received 50 μl of PBS solution with 1% DMSO containing 0.1% (v/v) terpinen-4-ol (1A).

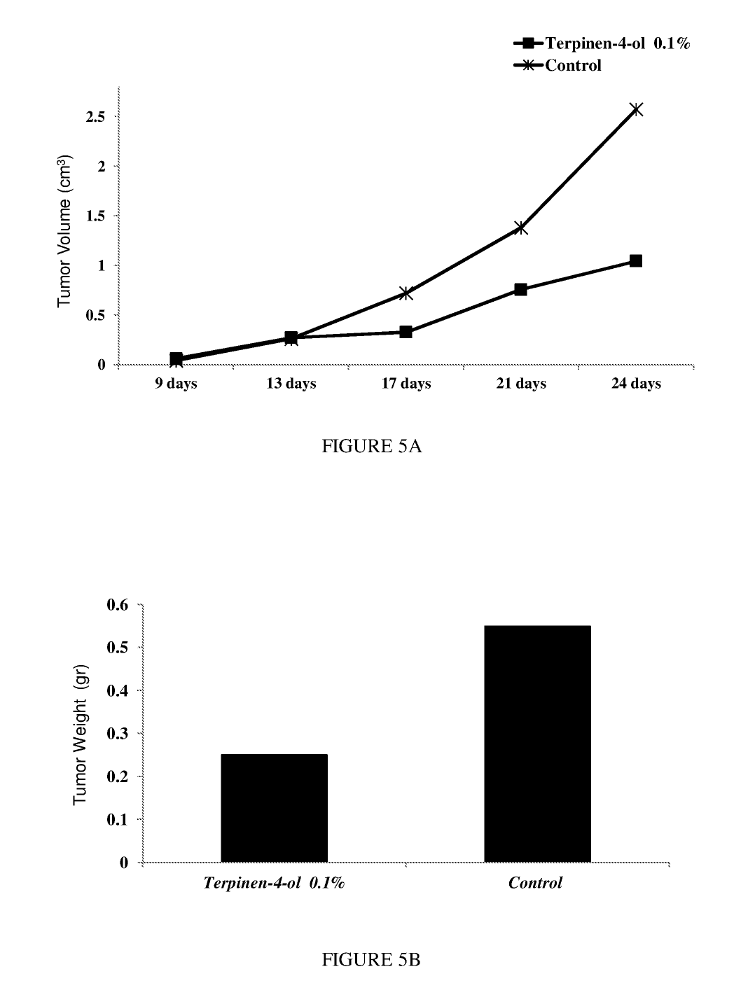

Mice were treated every second day. The mice were weighed and tumor volume was measured every three days (with a caliper) starting from the onset of treatment with terpinen-4-ol/control. Tumor volume was calculated as 4/3π·a·b². At the end of the experiment, the mice were sacrificed by cervical dislocation after anesthesia and the tumors were excised and measured for volume and weight. Half of each tumor was preserved in −80° C. and the other half was preserved in 4% formaldehyde. FIG. 5 clearly shows that administration of a 50 μl solution containing 0.1% terpinen-4-ol, which amounted to about 1.65 mg/Kg body weight per injection significantly reduced the volume and weight of the implanted tumor. These results show the capability of terpinen-4-ol to arrest tumor growth.

### Example 6

### Evaluation of a Combination Therapy of Terpinen-4-Ol and Immunotherapeutic Drug in Xenograft Model of Nude Mice Bearing DLD-1 Colorectal Cancer Cell Line

This study investigated the anti-cancer activity of terpinen-4-ol (1A) alone and in combination with the biologic agent cetuximub (Erbitox®).

Same xenograft model of nude mice bearing DLD-1 colorectal cancer cell line as described in Example 5 above was used. At the 11^{th} day after cell implantation, mice were randomly divided into groups and the treatment started. The chemotherapeutic agent (cetuximub, referred to hereinafter by its trade name Erbitox®) was administered via intraperitoneal (i.p) injection 24 hours before administration of terpinen-4-ol. The terpinen-4-ol was administered directly into the tumor (intra-tumor injection).

The treatment groups were as follows (4 mice per group): Control—1% DMSO in PBS; Erb—10 mg/Kg body weight of Erbitux®; 1A—50 μl 0.1% terpinen-4-ol dissolved in 1% DMSO in PBS; and Erb+1A—combination of Erbitux at 10 mg/Kg body weight and 0.1% terpinen-4-ol dissolved in 1% DMSO in PBS. Mice were treated every second day for 2 weeks. Mice were weighed and tumor volume was measured with a caliper before each additional treatment. Tumor volume was calculated as 4/3π·a·b².

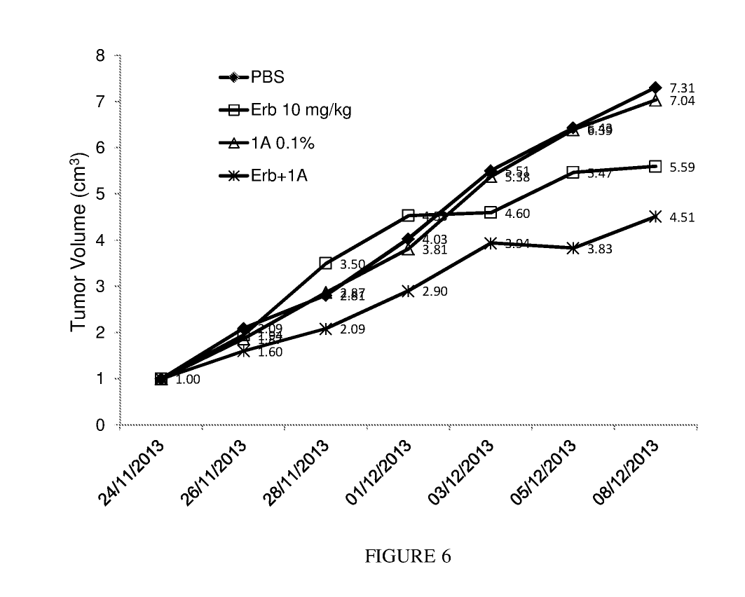

FIG. 6 shows the results obtained after two weeks of treatment. As is also shown in FIG. 5, at this time point administration of terpinen-4-ol had only minor effect on the tumor growth; however, the combination of terpinen-4-ol and Erbitux® resulted in significant reduction of the tumor volume compared to the reduction observed for each treatment alone. Furthermore, the combination activity was synergistic: while terpinen-4-ol reduced the tumor size (compared to control) by about 3.7% and Erbitux® by about 23.5%, the combination therapy reduced the tumor size (compared to control) by about 38.3%.

The assay is continued for additional 4-7 days. At the end of the experiment, the mice are sacrificed by cervical dislocation after anesthesia and the tumors are excised for further analysis.

### Example 7

### Evaluation of a Combination Therapy of Terpinen-4-Ol and a Chemotherapeutic/Biologic Drug

The xenograft model described in the method section hereinabove is used to examine further combinations of terpinen-4-ol and additional chemotherapeutic or immunotherapeutic drugs.

Cancer cells (selected from the cell types listed I in Table 1 hereinabove) are injected subcutaneously at one or two site on the back of the mice. When tumors are palpable (˜0.3-0.5 cm³), mice are randomly divided into groups and the treatment is started. Typically, terpinen-4-ol is injected directly to the tumor (i.t. injection) and the additional anti-cancer agent is administered by intraperitoneal (i.p) injection. The chemotherapy agent is injected 24 h before the injection of the terpinen-4-ol. The treatments administered are listed in Table 2 hereinbelow.

TABLE 2
Treatment examined by the in vivo xenograft model

| Treatment | Number of mice |
|---|---|
| PBS + 1% DMSO (Control) | 7 |
| 0.1% (or 0.06)% terpinen-4-ol | 7 |
| (in 1% DMSO in PBS) | |
| 0.1% (or 0.06)%)% terpinen- | 7 |
| 4-ol (in 1% DMSO in PBS) + | |
| Erbitux (10 mg/kg) | |
| Erbitux (10 mg/kg) | 7 |
| Oxaliplatin 5 mg/kg | 7 |
| 0.1% (or 0.06)%)% terpinen- | 7 |
| 4-ol (in 1% DMSO in PBS) + | |
| oxaiplatin (5 mg/kg) | |

Mice are treated every next day for 2-3 weeks. Mice are weighed and tumor volume is measured every three days (with a caliper) from the onset of Terpinen-4-ol treatment and measurement results are plotted. Tumor volume is calculated as 4/3π·a·b². At the end of the experiment, the mice are sacrificed by cervical dislocation after anesthesia and the tumors are excised and weighed.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-43. (canceled)

44. A method for treating cancer comprising administering to a subject in need thereof (a) an effective amount of terpinen-4-ol and (b) an effective amount of at least one additional anti-cancer agent to provide a combination therapy having an enhanced therapeutic effect compared to the effect of the terpinen-4-ol and the at least one additional anti-cancer agent each administered alone.

45. The method of claim 44, wherein the combination therapy has a synergistic therapeutic effect.

46. The method of claim 44, wherein the cancer is selected from the group consisting of solid tumors and non-solid tumors.

47. The method of claim 46, wherein the cancer is a solid tumor selected from the group consisting of colorectal cancer, gastric cancer, pancreatic cancer and prostate cancer.

48. The method of claim 44, wherein the at least one additional anti-cancer agent is selected from the group consisting of a chemotherapeutic agent and a biological agent.

49. The method of claim 48, wherein the at least one additional anti-cancer agent is a chemotherapeutic agent selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof.

50. The method of claim 48, wherein the at least one additional anti-cancer agent is a biological drug, said biological drug is an antibody selected from the group consisting of cetuximab (Erbitux®), anti-CD24 antibody and bevacizumab (Avastin®).

51. The method of claim 44, wherein the at least one additional anti-cancer agent is known to be effective in treating said cancer.

52. The method of claim 51, wherein the cancer is gastrointestinal cancer and the at least one additional anti-cancer agent is selected from the group consisting of oxaliplatin (Eloxatin®), fluorouracil (5-FU), anti-CD24 antibody, cetuximab (Erbitux®) and bevacizumab (Avastin®).

53. The method of claim 51, wherein the cancer is pancreatic cancer, and the at least one additional anti-cancer agent is selected from the group consisting of gemcitabine (Gemzar®) erlotinib hydrochlorides (Tarceva®) and humanized anti-CD24 monoclonal antibodies.

54. The method of claim 51, wherein the cancer is prostate cancer and the at least one additional anti-cancer agent is selected from the group consisting of cetuximab (Erbitux®), bevacizumab (Avastin®) and humanized anti-CD24 monoclonal antibodies.

55. The method of claim 44, wherein the terpinen-4-ol and the at least one additional anti-cancer agent are administered simultaneously, sequentially or concurrently.

56. A method for inhibiting cancer cell proliferation, comprising contacting cancer cells with therpinen-4-ol in combination with at least one additional anti-cancer agent, wherein the combination provides an enhanced anti-cancerous effect compared to the effect of the terpinen-4-ol and the at least one additional anti-cancer agent each administered alone.

57. A composition for treating cancer, the composition comprising a first component consisting of an effective amount of terpinen-4-ol and second component comprising an effective amount of at least one additional anti-cancer agent.

58. The composition of claim 57, wherein the collective amount of terpinen-4-ol and the at least one additional anti-cancer agent provides for an enhanced therapeutic anti-cancer effect.

59. The composition of claim 58, wherein the collective amount of terpinen-4-ol and the at least one additional anti-cancer agent provides for a synergistic therapeutic anti-cancer effect.

60. The composition of claim 57, wherein the at least one additional anti-cancer agent is selected from the group consisting of a chemotherapeutic agent and a biological agent.

61. The composition of claim 60, wherein the at least one additional anti-cancer agent is a chemotherapeutic agent selected from the group consisting of cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrxate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, erlotinib hydrochloride and combinations thereof.

62. The composition of claim 60, wherein the at least one additional anti-cancer agent is a biological drug, said biological drug is an antibody selected from the group consisting of cetuximab (Erbitux®), anti-CD24 antibody and bevacizumab (Avastin®).

63. The composition of claim 57, wherein the concentration of the at least one additional anti-cancer agent is selected from the group consisting of a concentration known to be used for treating said cancer or a lower concentration compared to the concentration known to be used for treating said cancer.
